(19) United States
(12) Patent Application Publication
(10) Pub. No.: US 2015/0209401 A1
(43) Pub. Date: Jul. 30, 2015

(54) Synergistic Multi-Active Composition for Topical Treatment of Disorders in Mammals, Use of Composition, and Method

(71) Applicant: Marco Antonio Botelho SOARES, Fortaleza (BR)

(72) Inventor: Marco Antonio Botelho SOARES, Fortaleza (BR)

(21) Appl. No.: 14/162,922

(22) Filed: Jan. 24, 2014

(51) Int. Cl. A61K 36/85; A61K 31/05; A61K 36/22

(52) U.S. Cl. CPC A61K 36/85; A61K 36/22; A61K 31/05

(57) ABSTRACT

A synergistic composition comprising two substrates, active against many diseases of mammals, particularly humans. The substrates are particularly extracts of the aroeira plant (Myracroduon urundeuva) and of the alecrim-pimenta plant (Lippia sidoides).

### FIELD OF THE INVENTION

The present invention relates to a synergistic composition comprising two plant substrates, active against many diseases of mammals, particularly humans.

### BACKGROUND OF THE INVENTION

Plants Lippia sidoides and Myracrodruon urundeuva are known in Brazilian folk medicine, traditionally used for treating bleeding gums and many other inflammatory diseases. They are found in the prior art references to the plant Myracrodruon urundeuva (Engl.), popularly known as “aroeira do sertao”, such as those mentioned below, typically concerning anti-inflammatory activity:

Teas: cuts and bruises, helping in healing;

Infusion for oral administration: inflammation of the liver and uterus;

Infusion: utero-vaginal douche for vaginal inflammation;

Postpartum seat bath;

Bark extract gargle: throat infections;

Stem bark powder: snake bite.

Lippia sidoides Cham. (Verbenaceae), popularly known as “alecrim pimenta” is largely grown in Northeastern Brazil. The hydro-alcoholic extract of this plant is largely used for the treatment of skin wounds, as mouth antiseptic, and in liquid soap preparations to treat and prevent general fungal infections of the body. Other uses mentioned below are typically related to antimicrobial activity: antiseptic, cleansing wounds, acne treatment, cold sore scab, white cloth, impinges, dandruff, foul smell of feet, armpits and groin, among others.

Surprisingly, it was found that compositions containing small amounts of these two combined substrates exhibit strong curative activity for various diseases, beyond what could be expected of antimicrobial and anti-inflammatory individual effects.

The thesis defended by Botelho, M. A. “The study of the effect of the gel of Alecrim-pimenta (Lippia sidoides) and Aroeira-do-sertao (Myracroduon urundeuva) and their isolated active principles in experimental periodontal disease”, Federal University of Ceará. Fortaleza 2007, Brazil, evaluated the effects of (a) individual extracts of each plant and their mixture and (b) gels with individual active ingredients contained in the two extracts, and the mixture of active ingredients with observed synergistic results upon microbial activity (bacteria and fungi) and rate of bone loss.

However, it was found that, unpredictably, the synergistic composition of that document is not in any way limited to just the disclosed applications.

Surprisingly, the effectiveness of combinations of such extracts or active principles contained therein also proved to be present in treating other diseases, injuries and traumas, without any relation to each other, providing new and strong evidence in all cases tested by topical application.

### SUMMARY OF THE INVENTION

The substrates are respectively the extracts of the aroeira plant (Myracroduon urundeuva) and of the rosemary-pepper plant (Lippia sidoides) popularly known as “alecrim-pimenta”. As used herein, mention to these substrates also refer to the main active ingredients contained in such plants.

Without limiting the invention to scientific explanations, this composition has an effect on the immune system of the patient in order to provide, as a whole, results that extend beyond the contribution of each individual component.

As the sense used herein, “topical treatment” includes any application upon skin or mucosa with transdermal or transmucosal action. Still according to the sense used herein, “disorders” includes diseases, trauma and injury to the mammalian body.

The effectiveness of the combination of the extracts or the active principles contained therein also proved to be effective in treating other diseases, injuries and traumas, among which are:

a. various treatments
Acne/Acne Rosacea
Folliculitis
Bedsores (patients in ICU)
Oral thrush
Acute pulpite
Halitosis
Dentin sensitivity
Post-dental surgical hemostasis
Dental alveolitis
Peri-implantitis (dental metallic implants)
Dental prosthesis candidiasis
Vaginal candidiasis
Cold sores (Herpes simplex)
Burns
Insects bites
Mycoses, including mycosis of nails
Athlete's Foot
Mycoses of dogs and cats
Allergic Dermatitis
Oral injuries (traumatic processes)
Radiomucositis (for patients with head and neck cancer)
Oro-pharyngeal candidiasis
Wound Healing/hemostasis in skin lacerations
Hemorrhoid

B. other applications
Prevention of foul odor in the feet and armpits
Irrigant for treatment and disinfection of dental channel
Normal and laser hair removal anti-oxidant gel
Aftershave GEL
Removal of warts (papillomas)
Healing (surgical and traumatic wounds)
Prevention of keloid formation (plastic surgery)
Muscle Injuries
Physiotherapy gel (along with ultrasound device)
Transmucosal and transdermal hormone delivery

### BRIEF DESCRIPTION OF THE DRAWINGS

FIG. 1A shows the condition of the skin before treatment as set forth in Example 2;

FIG. 1B shows changes after 24 h with three applications per day as set forth in Example 2;

FIG. 1C shows the results after 1 week, 3 applications per day as set forth in Example 2;

FIG. 2A shows the initial condition of the patient as set forth in Example 3;

FIG. 2B shows the effect 3.5 hours after the first application as set forth in Example 3;

FIG. 2C shows the results after 24 hours as set forth in Example 3;

FIG. 3A shows the site of the burn as set forth in Example 4;

FIG. 3B shows the results after application to the injured area with healing without the formation of bubbles as set forth in Example 4;

FIG. 4A shows the initial state (arrow indicates injury) as set forth in Example 5;

FIG. 4B shows the result after 48 hs as set forth in Example 5;

FIG. 5A shows the state before treatment as set forth in Example 6;

FIG. 5B shows the appearance after treatment as set forth in Example 6;

FIG. 6A shows the state of the cut before the treatment as set forth in Example 7;

FIG. 6B shows the appearance after 48 h as set forth in Example 7; and,

FIG. 6C shows the appearance after 72 h as set forth in Example 7.

### DETAILED DESCRIPTION OF THE INVENTION

It is important to note that, unlike a purely additive effect of a substrate with antibacterial capacity and another substrate with anti-inflammatory capacity, it was found a synergistic effect, with performance and results above the mere addition of the individual effects. Additionally, this effect was repeatedly seen in use in many other conditions, diseases, injuries and traumas.

Thus, the present invention of multi-active synergistic composition for topical treatment of disorders in mammals, is characterized by comprising the combination of an extract of the aroeira plant (or isolated compounds found in extracts of the aroeira plant), a plant extract of alecrim-pimenta (or compounds isolated from plant extracts found in alecrim-pimenta), and one or more pharmaceutically acceptable excipients.

The “Aroeira” is a plant also known by the names of aroeira do campo, aroeira do sertão, aroeira da serra, urundeúva, uriunduba e aroeira preta, corresponding to the botanical classification M. urundeuva all Br (previous designations being Astronium juglandifolium and Astronium urundeuva).

The “Alecrim-pimenta” is a plant also known by the names of alecrim-grande, estrepa-cavalo, alecrim-bravo, alecrim do nordeste and lipia, corresponding to the botanical classification Lippia sidoides Cham.

The “extract of the aroeira plant” refers in general to extracts containing chalcones, tannins, flavonoids and alkaloids, of fractions or extracts containing mixtures thereof, for example, glycolic, butanolic, ethanolic extracts or fluid extract. It also refers to one or more of the dimeric chalcones urundeuvine A, B or C, regardless of their origin in this plant.

The “extract of the alecrim-pimenta plant” refers generally to extracts containing predominantly carvacrol and thymol, or extract fractions enriched in thymol and/or carvacrol, the thymol compound per se, the carvacrol compound per se, or mixtures thereof, regardless of its origin is the alecrim-pimenta plant. Example of a suitable extract is the essential oil obtained from the leaves of this plant.

The compositions of the invention may be formulated in any appropriate physical form, for example: powder, mud, gel, ointment, solutions, dispersions, emulsions, soaps, douches, patches, stickers, etc. A person skilled in the art knows how to formulate such compositions containing pharmaceutically acceptable excipients, for example, selected from among those cited in the following literature: Remington's Pharmaceutical Sciences, Mack Publishing, the European or the Brazilian Pharmacopeias.

In a particular embodiment, the invention encompasses the use of a carrier in the form of micro or nanogel.

Besides the mentioned synergistic mixture, the compositions of the invention may contain active ingredients and other ingredients, aimed to provide specific effects. A purely exemplary and non-limiting list includes one or more actives among healing agents, antibiotics, analgesics, hormones, haemostatic, antiseptic agents, antioxidants, antifungal, anti-inflammatory, anti-allergic.

### EXAMPLES

The following examples are particular embodiment of the inventions, given only for illustrative purposes, without imposing limitations beyond those contained in the claims presented farther on.

### Example 1

### Preparation of a Composition Containing Nanogel the Invention

### B. Formulation

| | | |
|---|---|---|
| | CONCENTRATION | |
| NAME OF COMPONENT | (% W/W) | SUPPLIER |

PHASE A

| | | |
|---|---|---|
| BHT (Butil- | 0.2 | Braskem |
| hidroxitoluene) | | |
| Thymol | 0.12 | Gerbras |
| Sodium Metabissulfite | 0.5 | Gerbras |
| Propylene glycol | 2 | Galena |

PHASE B

| | | |
|---|---|---|
| Carbopol gel | Qsp (100 gr) | Preparation |
| | | described on item B |

PHASE C

| | | |
|---|---|---|
| Glycolic Extract from | 5 | Mapric |
| Aroeira | | |
| Mixture of parabens | 0.25 | Generic |
| Edulcorant | 3.2 | Any |

### Preparation

### Phase A

In a gral mix thymol and sodium metabisulfite
Add and mix propylene glycol

### Phase B

Weigh and add carbopol gel to phase A to solubilization

### Phase C

Prepare mixture of glycolic extract of aroeira, antimicrobial solution sweetener, add to earlier stage phases and mix in a roller mill.
Adjust the pH to between 5.5 and 7.0

### B. Preparation of Carbopol Gel

GEL COMPONENTS

| | CONCENTRATION | |
|---|---|---|
| NAME OF COMPONENT | (% W/W) | SUPPLIER |
| Carbopol 940 | 1.8 | FAGRON |
| mixture of parabens | 0.25 | Generic |
| Imidazolidinyl urea | 0.3 | Germall 115 |
| | | Mapric |
| aminomethylpropanol | 0.75 | AMP 95 |
| | | Galena |
| Demineralized water | qsp (100 g) | |

### Preparation of Gel

Add imidazolydinyl urea to a becker containing demineralized water until solubilization, and then add the antibacterial.
Slowly Add carbopol 940 until complete solubilization;
Slowly add the aminomethylpropanol until a smooth and homogeneous gel is obtained;
Adjust the pH to between 5.5 and 7.0.

### Example 2

### Acne Treatment.

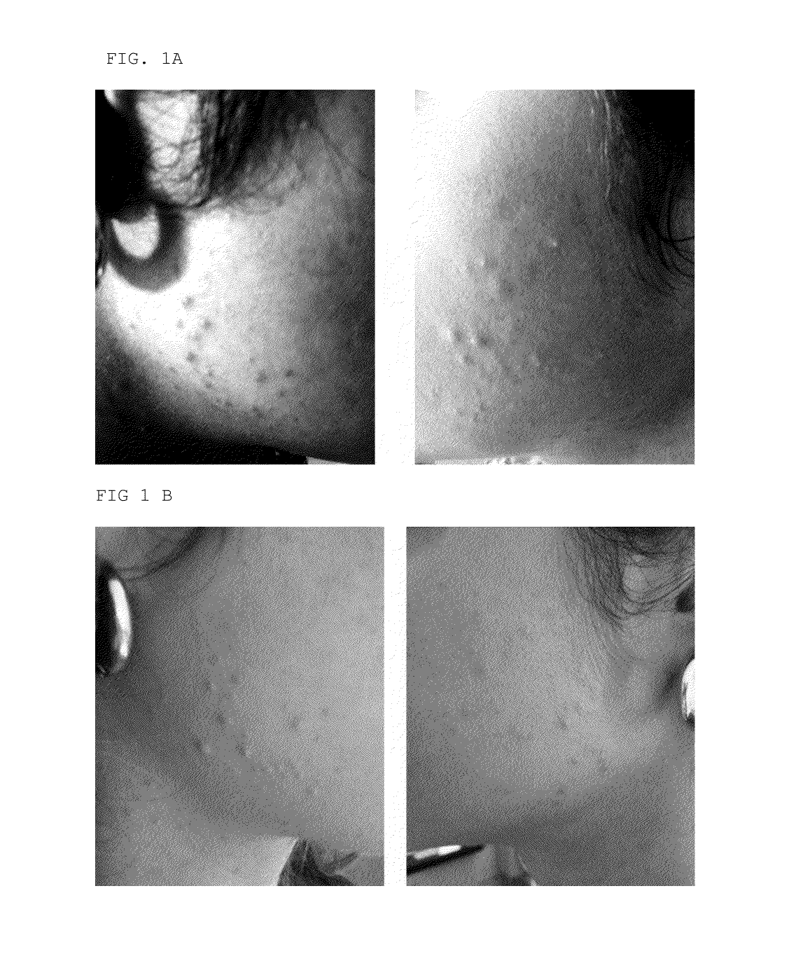

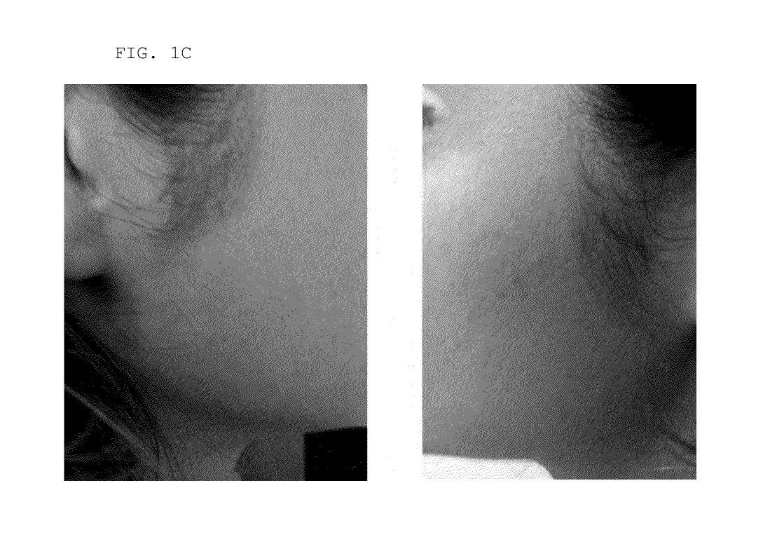

The gel of example 1 was applied to a patient twice a day over one week. FIG. 1A shows the condition of the skin before treatment, FIG. 1B shows changes after 24 h with three applications per day, and FIG. 1C shows the results after 1 week, 3 applications per day.

A faster and more effective improvement was found when compared to other medications commonly indicated for the treatment of such pathology (benzoyl peroxide, retinoic acid, azelaic acid).

### Example 3

Treatment of cold sores (herpes simplex).

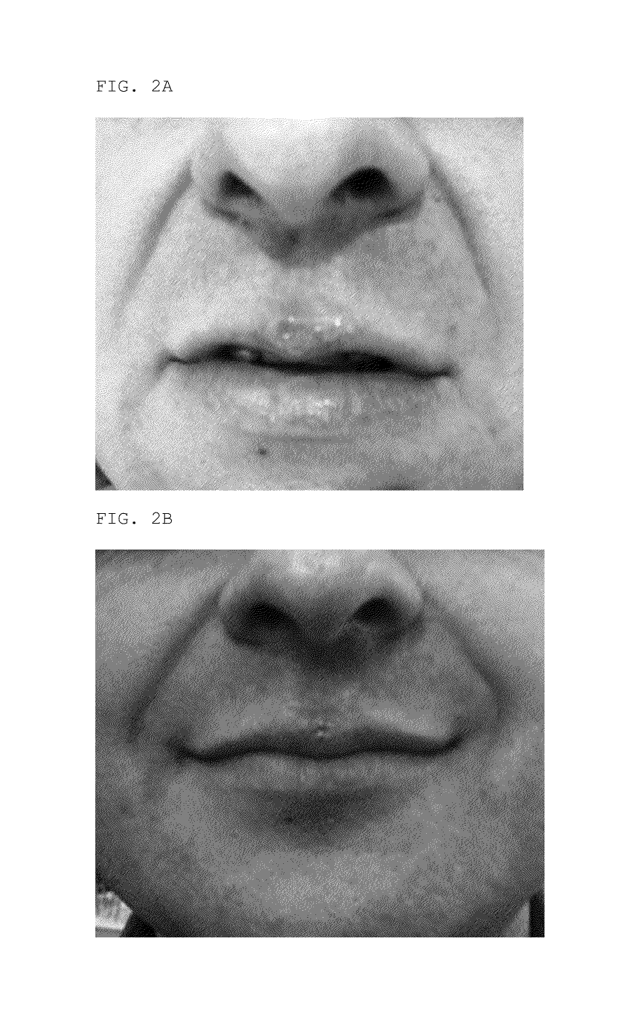

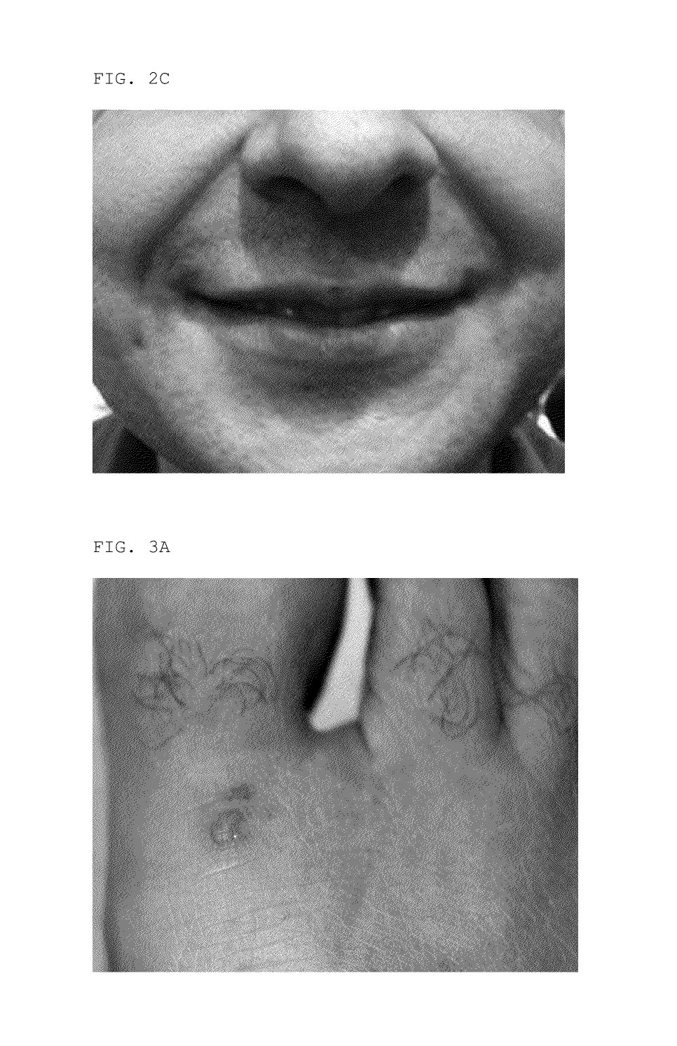

The gel from example 1 was applied 3 times a day. A significant improvement was found, including the decrease of the pain and the course of the disease. FIG. 2A shows the initial condition of the patient, FIG. 2B shows the effect 3.5 hours after the first application and FIG. 2C shows the results after 24 hours. The gel showed a faster and more efficient activity when compared to acyclovir (gold standard drug for the treatment of herpes labialis).

### Example 4

Hot vegetable oil skin Burn.

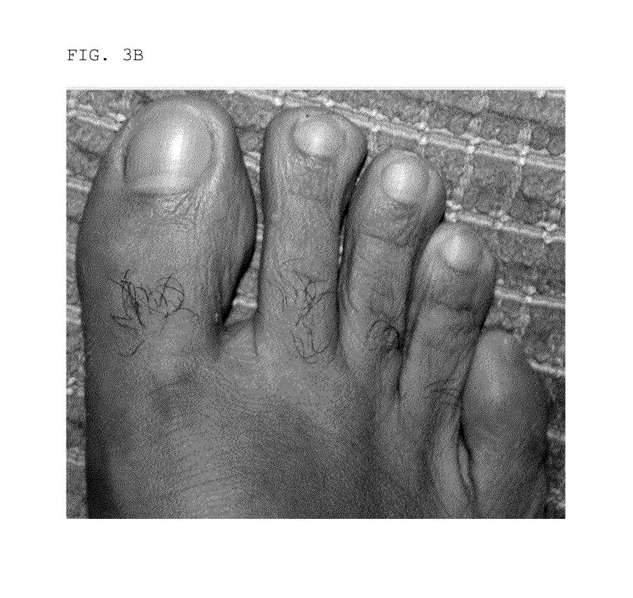

The gel of Example 1 was applied to the injured site at the foot of a patient, over a week, three times a day. FIG. 3A shows the site of the burn, and FIG. 3B shows the results after application to the injured area with healing without the formation of bubbles.

### Example 5

Oral aphthae (oral canker).

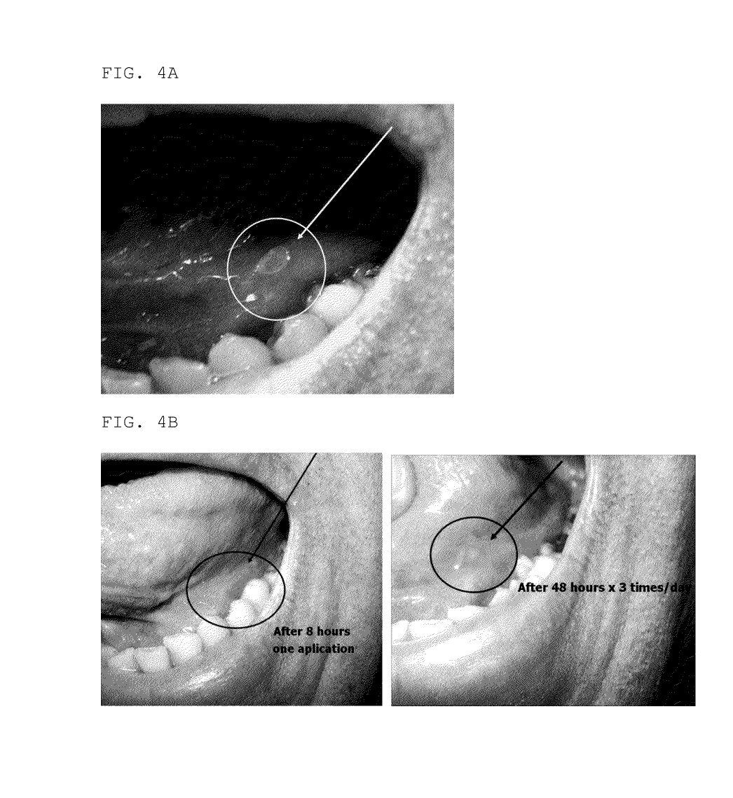

The gel of example 1 was applied to the tongue of a patient, twice a day during 48 hours. FIG. 4A shows the initial state (arrow indicates injury), and FIG. 4b shows the result after 48 hs.

### Example 6

Shaving ANTI haemostatic and anti-irritating.

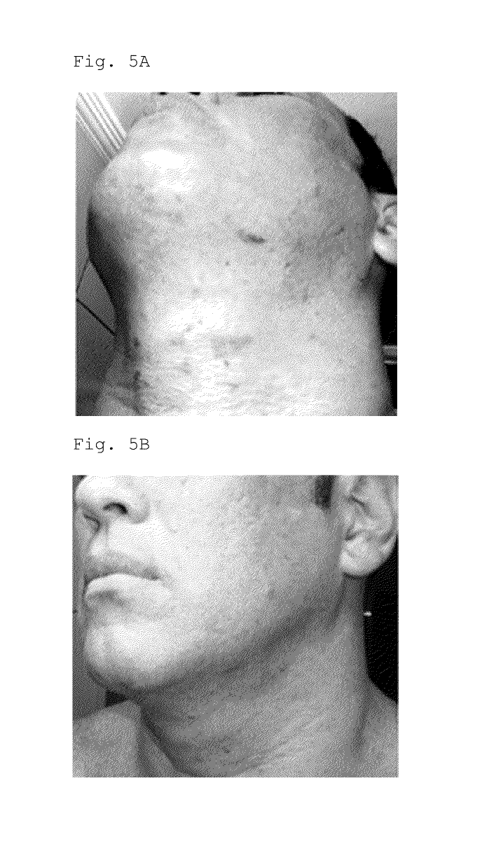

The gel of example 1 was applied to the shaved area of a patient, just once after shaving. FIG. 5A shows the state before treatment and FIG. 5B shows the appearance after treatment. There was remarkable improvement in inflammation of the injured area, and the patient reported calming action after gel application.

### Example 7

Cut healing.

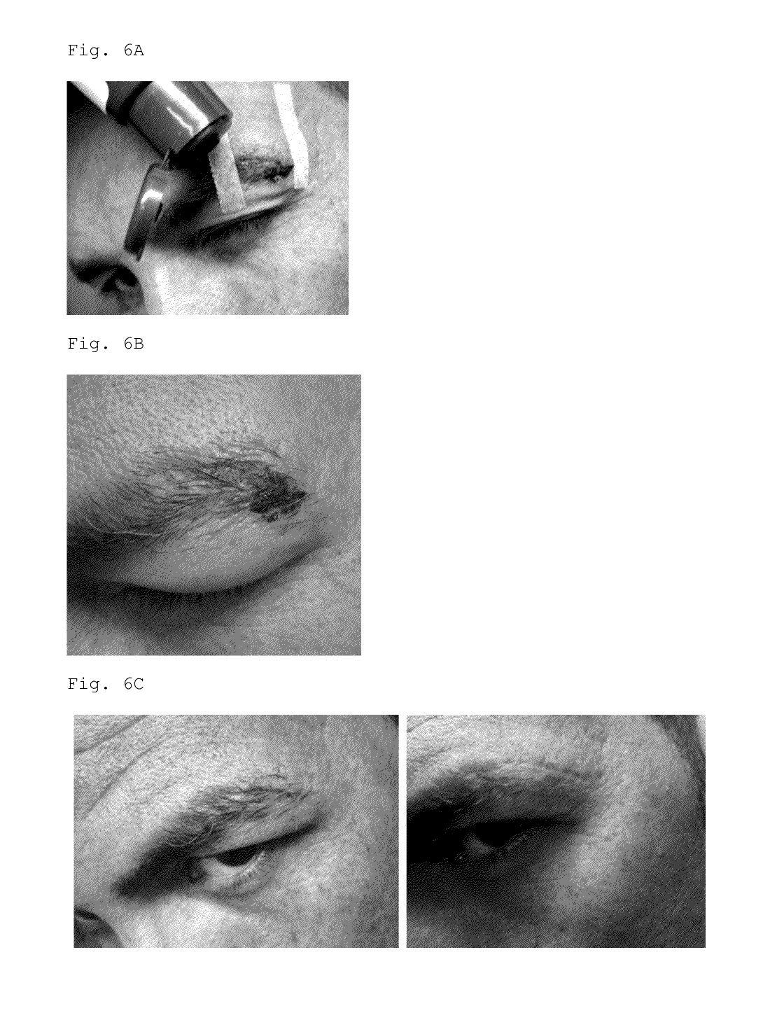

The gel of example 1 was applied to a superciliary cut of a patient, 3 times a day over 72 hours. FIG. 6A shows the state of the cut before the treatment, FIG. 6B shows the appearance after 48 h and FIG. 6C shows the appearance after 72 h.

With the information and examples herein a person skilled in the art is able to carry out equivalent forms of the invention, not expressly described here, but with functions and results of the same nature as the ones described, therefore within the scope of the appended claims.

1. A multi-active synergistic composition for topical treatment of disorders in mammals, comprising an extract of aroeira plant, an extract of alecrim-pimenta plant and one or more pharmaceutically acceptable excipients.

2. The composition according to claim 1, wherein the extract of aroeira plant comprises chalcones, alkaloids, tannins and flavonoids.

3. The composition according to claim 2, wherein the aroeira plant extracts are selected from the group consisting of: glycolic extract, butanolic extract, ethanol extract and fluid layers.

4. The composition according to claim 2, wherein the plant extract aroeira comprises one or more dimeric chalcones among urundeuvine A, B and C.

5. The composition according to claim 1, wherein the extract of the alecrim-pimenta plant is an essential oil obtained from leaves.

6. The composition according to claim 1, wherein the extract of alecrim-pimenta plant comprises mainly thymol and carvacrol.

7. The composition according to claim 1, wherein the extract of alecrim-pimenta plant is selected from the group consisting of: thymol compound, carvacrol compound, and mixtures thereof.

8. The composition according to one of claim 1, formulated as a powder, slurry, gel, cream, ointment, solution, dispersion, emulsion, soap, douche or adhesive patch.

9. The composition according to claim 8, formulated as micro or nano emulsion.

10. The composition according to claim 1, for use in medical, dental or veterinary therapy.

11. Use of the composition of claim 1 for the preparation of a product for topical treatment of diseases.

12. Use of the composition of claim 1 for the preparation of a product for topical treatment of one or more of the following conditions: acne, folliculitis, sores, mouth ulcer, acute pulpitis, the dentin sensitivity, post-dental surgical haemostasis, dental alveolitis, peri-implantitis, dental prosthesis candidiasis, vaginal candidiasis, herpes simplex, cold sores, burns, insect bites, ringworm, fungal infections of dogs and cats, allergic dermatitis, oral cuts, radiomucosite, oro-pharyngeal candidiasis, hemostasis cuts and wounds, hemorrhoids, halitosis.

13. Use of the composition of claim 1 for the preparation of a product for topical use in one or more of the following applications: prevention of foul odor in the foot or armpits, irrigant for treatment and disinfection of dental channel, anti-oxidant gel, aftershave gel, removal of warts, healing, prevention of keloid formation, muscle, physiotherapy bruises with +ultrasound gel, transmucosal and transdermal hormone delivery.

14. A method of using the composition of claim 1 for the therapeutic treatment of one or more of: acne, folliculitis, sores, mouth ulcer, acute pulpitis, the dentin sensitivity, hemostasis post-dental surgery, dental alveolitis, peri-implantitis, candidiasis dental prosthesis vaginal candidiasis, herpes simplex, cold sores, burns, insect bites, ringworm, fungal infections of dogs and cats, allergic dermatitis, oral cuts, radiomucosite, oro-pharyngeal candidiasis, hemostasis in cuts and wounds, hemorrhoids, halitosis, foul odor prevention in feet or armpits, irrigating treatment and disinfection of dental gel anti-oxidant channel, aftershave gel, removal of warts, healing, prevention of keloid formation, muscle injuries, physical therapy with ultrasound+gel, transmucosal and transdermal hormone delivery.
